(19) United States
(12) Patent Application Publication
(10) Pub. No.: US 2002/0004053 A1
(43) Pub. Date: Jan. 10, 2002

(54) Cellular or acellular organism eradication via photodynamic activation of a cellular or acellular organism specific immunological response

(76) Inventor: Merrill A. Biel, Mendota Heights, MN (US)

Correspondence Address: John F. Klos,, Larkin, Hoffman, Daly & Lindgren, Ltd, Suite 1500, 7900 Xerxes Avenue South, Bloomington, MN, 55431-3333, US

(21) Appl. No.: 09/782,841

(22) Filed: Feb. 14, 2001

Related U.S. Application Data
(63) Continuation-in-part of application No. 09/139,861, filed on Aug. 25, 1998.

(51) Int. Cl. A61K 39/00; A61N 1/30

(52) U.S. Cl. 424/277.1; 604/20

(57) ABSTRACT

A method of eradicating cellular or acellular organisms of a living body by promoting a systemic immunologic activity specific to the cellular/acellular organisms including the steps identifying a cellular/acellular organism site of the living body; combining an administration of an immunologic adjuvant to said body, the immunologic adjuvant promoting a systemic increased level of molecular and cellular factors and cells necessary to effect a systemic immunologic response and an administration of a photodynamic light therapy proximate said cellular/acellular organism site, said photodynamic light therapy having a sufficient light dosage to cause cell necrosis of a cellular/acellular organism within the cellular/acellular organism site, said photodynamic cellular/acellular organism necrosis resulting in the release of necrosis-generated cellular/acellular organism antigens; and promoting and enhancing a systemic cellular/acellular organism-related immunologic response of said body as a result of an interaction between the molecular and cellular factors and cells and the necrosisgenerated cellular/acellular organism specific antigens, said systemic immunologic response yielding an increased level of cellular/acellular organism specific antibodies and other immunologic cellular/acellular organism specific products and cells specifically targeted to immunologically eradicate similar cellular/acellular organisms.

### CROSS REFERENCE TO RELATED APPLICATIONS

This application claims the benefit of priority pursuant to 35 USC § 120 from U.S. patent application Ser. No. 09/139,861 on Aug. 25, 1998.

### BACKGROUND OF THE INVENTION

1. Field of the Invention

Photodynamic therapy (PDT) utilizes light energy in combination with photosensitizing agents to treat or detect pathologies of living tissue, including cancer and microbiological pathogens. Once pre-sensitized by the photosensitizing agent, the cancerous or abnormal cells can be eradicated with light of an appropriate wavelength or waveband corresponding to an absorbing wavelength of the agent, with minimal damage to normal tissue.

Specific immunotherapy utilizes a systemic immunological response to target specific foreign cells. Specific immunity is an immune status in which there is an activity directed solely against the antigenic determinants that stimulated it. It may be active and specific as a result of naturally acquired infection or intentional vaccination.

2. Brief Discussion of the Prior Art

Photodynamic therapy, PDT, involves the treatment of diseased tissue using photosensitizing chemicals and light. PDT, as presently used, is based on the observation that certain photosensitizing compounds preferentially concentrate in abnormal tissue or cells or organisms relative to most other normal tissues or cells or organisms. A well known example of a procedure which exploits this differential concentration of photosensitizer is the use of PDT to treat tumors. This preferential concentration, or therapeutic ratio as it is sometimes called, is the basis of obtaining the potential therapeutic selectivity that is usually desired in the clinical application of PDT. This is generally obtained by first administering the photosensitizer by a suitable route, generally intravenously, then waiting for some period of time for the photosensitizer to be accumulated within the target tissues. Selective affinity and retention of photosensitizers in hyperproliferating tissue or cells or organisms has been documented for a variety of photosensitizers.

PDT is generally used to treat hyperproliferating tissues, i.e. cancer, etc, by first administering a photosensitizer to the patient by a suitable route such as, but not limited to, intravenous [IV], intramuscular [IM], intraperitoneal [IP] injection, topical, and oral administrations, and then waiting for a predetermined period of time known to be sufficient to effect the preferential uptake and retention of the photosensitizer in the target tissue relative to the concentration of the photosensitizer in normal (non-hyperproliferating) tissues. By permitting time to elapse after systemic administration of the drug, the photosensitizer is generally localized in a variety of tissue/cell types as well as locations within the target tissue. The time for photosensitizer build-up in a target tissue varies but is in the range of 2-96 hours. The resulting therapeutic response therefore generally involves a variety of cytological effects.

Photodynamic therapy is a treatment that is based upon the differential uptake by cellular or acellular organisms of photosensitizing agents, followed by irradiation of the cellular or acellular organisms to cause a photochemical reaction that is believed to generate chemically disruptive species, such as singlet oxygen, without increasing the temperature of the treated tissue. These disruptive species in turn injure the cells through reaction with cell parts, such as cellular and nuclear membranes. Photodynamic therapy has been used successfully for treating several types of cancer cells.

Specific immunity is an immune status in which there is an altered reactivity directed solely against the antigenic determinants (infectious agent or other) that stimulated it. It is sometimes referred to as acquired immunity. It may be active and specific, as a result of naturally acquired (apparent or inapparent) infection or intentional vaccination. Cellular components include the lymphocyte (e.g., T-cells, B-cells, natural killer (NK) cells), and immunoglobulins as the soluble component. The action of T-cells and NK-cells in recognizing and destroying foreign cells or cell components is termed cell-mediated immunity.

### SUMMARY OF THE INVENTION

The present invention is method for eradicating primary and metastatic tumors via a photodynamic activation of a tumor specific immune response. Another aspect of the present invention is a method for eradicating cellular and acellular organisms, such as bacteria, fungi, parasites, viruses. According to one embodiment of the present invention, a treatment method includes a combined administration of a photodynamic therapy of a primary tumor site and an administration of an immunologic adjuvant. The timing and sequence of the photodynamic therapy and adjuvant administration may be varied, depending on the particular treatment needs (e.g., cell type, location, mass, etc.) Another aspect of the present invention involves the administration of a photosensitizing agent into or near a cellular or acellular organism site. The cellular or acellular organism site is then lased at an irradiance sufficient to induce cellular or acellular organism necrosis, said photodynamic cellular or acellular organism necrosis resulting in the release of cellular or acellular organism specific antigens from the necrosed cell or acellular organism. As a result, in the presence of the adjuvantenhanced immune system a cellular or acellular organism specific enhanced systemic immunologic response of said body results from an interaction between the immunologic molecular and cellular factors and cells and necrosis released cellular or acellular organism antigens. The cellular or acellular organism specific enhanced systemic immunologic response results in an increased level of cellular or acellular organism specific antibodies and other immunologic anti-cellular or acellular organism specific products and cells specifically targeted to eradicate similar cellular or acellular organisms.

In accordance with one aspect of the invention, an administration of an immunologic adjuvant precedes the administration of the photodynamic light therapy. In other preferred methods of practicing the invention, the administration of the adjuvant may occur during or after the administration of the photodynamic light therapy. The adjuvant may be administered at routine intervals after the photodynamic light therapy to sustain the immunologic cellular or acellular organism specific response.

In accordance with another aspect of the invention, a primary tumor cell site, such as a malignant tumor, is administered a solution containing a photosensitizing agent and an immunoadjuvant. The tumor cell site is then illuminated with low energy laser emitting a wavelength of radiation complementary to that of the photosensitizing agent. The photodynamic illumination induces non-thermal cellular destruction and stimulates the self-immunological defense system against targeted metastatic tumor cells.

In accordance with another aspect of the invention, an immune modulator may be administered to the body before, during, or after the photodynamic light therapy. The immune modulator may be administered on a regular basis after PDT treatment in order to continue to promote a tumor cell specific systemic immunologic response.

The present invention has several advantages over other treatment modalities. The most significant advantage is a combined primary and metastatic tumor destruction. The primary tumor loss is caused by photodynamic eradication. When photodynamic destruction occurs, the fragmented cell and cellular molecules, including necrosis-released cell antigens, are disbursed within the host in the presence of the immunologically potentiating material, which may be administered before, during, or after the photodynamic primary tumor cell eradication. As a result, an in situ vaccine is formed. There follows an immediate mobilization of cell-mediated immunity which encompasses NK-cells and recruited killer T-cells. These cells migrate to the sites of similar antigens or chemicals. In time, an increase in cytotoxic antibodies is present. These antibodies circulate within the body and attach to cells and materials for which they have been encoded. If this attachment occurs in the presence of complement factors, the result is cellular death.

In sum, primary and metastatic cancer eradication can be achieved via methods of the present invention as a result from the photodynamic eradication of the primary tumor cells and an enhanced immune system response targeting the metastatic tumor cells. An immunologic adjuvant may be introduced at single or multiple administrations before, during, or after the photodynamic eradication of the primary tumor cells. Booster administrations of the adjuvant may take place after the administration of the photodynamic light therapy.

Still other objects and advantages of the present invention will become readily apparent to those skilled in this art from the following detailed description, wherein there is shown and described only the preferred embodiments of the invention, simply by way of illustration of the best mode contemplated for carrying out the invention. As will be realized, the invention is capable of modifications in various obvious respects, all without departing from the invention. Accordingly, the description should be regarded as illustrative in nature, and not as restrictive.

### DETAILED DESCRIPTION OF THE PREFERRED EMBODIMENTS

Photodynamic cancer cell therapy is a treatment that is based upon the differential uptake by cancerous cells of photosensitizing agents, followed by light irradiation of the cells to cause a non-thermal photochemical reaction that is believed to generate chemically disruptive species, such as singlet oxygen. These disruptive species in turn destroy the cells through reaction with cell parts, such as cellular and nuclear membranes. Photodynamic necrosis of tumor cells results in the release of antigens and other cellular material specific to the tumor cells. Photosensitizers including PHOTOFRIN, SNET2, FOSCAN, methylene blue, and toluidene blue are relatively selectively retained in tumor tissue due to increased uptake of the drug in mitochondria, abnormal tumor vasculature, the nucleus, and macrophages. The irradiation of cells may be at a light wavelength from about 400 nm to about 800 nm, a light dosage range from about 10 J/cm² to about 250 J/cm², and a light dosage rate ranges from about 50 mw/cm² to about 200 mw/cm².

Immunologic adjuvants, such as DETOX (RIBI Pharmaceuticals), GM-CSF, GCSF, etc., may be administered to heighten or enhance the nonspecific immune system of the body by increasing the level of nonspecific immune-related molecular and cellular factors and cells. In the presence of tumor cell-specific antigens the enhanced level of nonspecific immune related molecular and cellular factors and cells yields an increased level of tumor cell-specific antibodies and other immunologic tumor cell-specific products and cells for eradicating specific tumor cells.

The method of treating a living body having a primary tumor and a metastatic tumor(s) can be enhanced by combining the two modalities. A method of treating a primary and metastatic tumor may include the following steps: identifying a tumor cell site, administering an immunologic adjuvant; waiting a time period, such as 2 to 10 days for an immune response; administering photosensitizing agents; and, administering photodynamic light therapy with light energy source.

Yet another method of treating a primary and metastatic tumor may include the following steps: identifying a primary tumor cell site; administering a photodynamic therapy at the primary tumor cell site to eradicate tumor cells and release tumor cell specific antigens; and administering an immunologic adjuvant simultaneously with or after the photodynamic therapy. The immunologic adjuvant may be administered at a standard concentration for immunization procedures of the body, and may include one or more booster administrations as appreciated by those skilled in the relevant arts.

The immunologic adjuvant may be administered to the body via a variety of known pharmaceutical administration approaches, including but not limited to a direct injection into the tumor, an intravenous administration, etc. Adjuvant concentrations may be standard concentrations for immunization procedures. Examples of immunologic adjuvants include: Freund's complete/incomplete adjuvant, DETOX by Ribi Pharmaceutical, Inc., granulocyte colony stimulator factor (G-CSF), and granulocyte macrophage colony stimulator factor (GM-CSF), and other biological enhancers.

Modulators of the immune response may also be administered to the body to augment the tumor specific systemic immunologic response in the presence of PDT generated tumor antigens and the heightened nonspecific systemic immune response generated by the administration of the immunologic adjuvant. Immune modulators (IMs) of the immune response include cytokines, including interleukins (1-15), interferon, particularly interferon gamma, tumor necrosis factors (TNF) alpha and beta, angiogenesis factors, integrins and matrix metalloproteinases in particular 1,3,8,9. These may be administered before, during or after PDT or before, during or after adjuvant administrations. The immune modulators may be administered intravenously, subcutaneously, intratumorally, or via other known administration approaches. The IMs may be administered on a regular basis after PDT treatment in order to sustain a tumor cell specific systemic immunologic response.

In one preferred method of practicing the invention, the time period between immunologic adjuvant administration and commencement of photodynamic therapy may vary depending on the particular immunologic adjuvant, and may be between 2 to 10 days. In other preferred embodiments of practicing the invention, the photodynamic therapy may occur simultaneously with or precede the administration of the immunologic adjuvant.

In other preferred embodiments of the invention, the tissue site may contain cellular and/or acellular organisms, such as fungi, bacteria, viruses, etc. Eradication of the cellular and/or acellular organisms may be provided via a cell or acellular organism specific immune response achieve by: identifying a cell or acellular organism site, administering an immunologic adjuvant; waiting a time period, such as 2 to 10 days for an immune response; administering photosensitizing agents; and, administering photodynamic light therapy with light energy source.

Yet another method of treating an organism site via an organism specific immune response may include the following steps: identifying an organism site; administering a photodynamic therapy at the organism site to eradicate some of the organisms and release organism specific antigens; and administering an immunologic adjuvant simultaneously with or after the photodynamic therapy. The immunologic adjuvant may be administered at a standard concentration for immunization procedures of the body, and may include one or more booster administrations as appreciated by those skilled in the relevant arts. Additionally, an immune modulator(s) may be administered after the photodynamic therapy for a period of time necessary to sustain an organism specific immune response.

Particular examples of methods of treatment according to the present invention follow. These examples are not intended to limit the scope of the present invention, but provide more detailed description of preferred embodiments of the invention.

The present invention provides a method of treating a living body having an infectious process or a primary tumor and a metastatic tumor, said method including the steps of:

(1) identifying a primary tissue site (cancer, fungal infection, acellular organism site, viral infection, bacterial infection, parasitic infection, etc.) of the living body;

(2) combining an administration of an immunologic adjuvant at a predetermined concentration to said body, said predetermined concentration being approximately a standard concentration for immunization procedures of said body, said administered immunologic adjuvant resulting in a systemic condition of heightened nonspecific enhanced immune system of the body, including an increased level of nonspecific immune-related molecular and cellular factors and cells, and an administration of a photodynamic light therapy proximate said primary tissue site, said photodynamic light therapy having a light wavelength and a sufficient light dosage to eradicate cells and/or acellular organisms within the primary tissue site, said eradicated primary tissue site cells and/or acellular organism releasing necrosis-related cell or acellular organism specific antigens; and

(3) promoting and enhancing a systemic immunologic response of said body as a result of an interaction between the increased level of nonspecific immune-related molecular and cellular factors and cells and photodynamic light therapy released cell or acellular organism specific antigens, said systemic immunologic response yielding increased levels of cell or acellular organism specific antibodies and other immunologic cell specific products and cells for eradicating cells relating to the primary tissue site.

The step of administering an immunologic adjuvant to the body can be made via a variety of known administration approaches, including an intratumoral injection, intravenous [IV] injection, intramuscular [IM] injection, intraperitoneal [IP] injection, topical administration, and/or an oral administration.

The step of administering an immunologic adjuvant to the body may include administration before, during, and/or after the step of administering the photodynamic light therapy.

The step of administering an immunologic adjuvant may include administrations provided at intervals after the photodynamic light therapy. Similarly, the step of administering the photodynamic light therapy may include administrations provided at intervals after one or more administrations of the immunologic adjuvant. Repeat administrations of the photodynamic therapy and/or immunologic adjuvant at intervals may promote a sustained cell/organism specific immunologic response.

The method may also include an administration to the body either singularly, or at intervals, of an immune modulator in order to continue to promote a cell specific systemic immunologic response.

G-CSF Adjuvant

1. Administer adjuvant compound subcutaneously at 5 μgram/kg/day; and

Wait 2-5 days after administration of immunologic adjuvant before photodynamic therapy.

2. Administer a photodynamic therapy;

Wait 2-5 days after administration of the photodynamic therapy; and

Administer the adjuvant compound subcutaneously at 5 μgram/kg/day;

3. Simultaneously administer a photodynamic therapy and the adjuvant compound subcutaneously at 5 μgram/kg/day.

GM-CSF Adjuvant

1. Administer compound intravenously at 250 μgram/m²/day; and

Wait 2-10 days after administration of immunologic adjuvant before

2. Administer a photodynamic therapy;

Wait 2-10 days after administration of the photodynamic therapy; and

Administer the adjuvant compound intravenously at 250 ugram/m²/day;

3. Simultaneously administer a photodynamic therapy and the adjuvant compound subcutaneously at 250 μgram/m²/day.

Freunds' Complete/Incomplete Adjuvant

1. Administer compound intratumorally at 1-10 cc; and

Wait 2-9 days after administration of immunologic adjuvant before photodynamic therapy.

2. Administer a photodynamic therapy;

Wait 2-9 days after administration of the photodynamic therapy; and

Administer the adjuvant compound intratumorally at 1-10 cc;

3. Simultaneously administer a photodynamic therapy and the adjuvant compound subcutaneously at 1-10 cc.

DETOX Adjuvant (RIBI Pharmaceutical)

1. Administer compound intratumorally at 1 cc/cm²; then

Wait 2-9 days after administration of immunologic adjuvant before photodynamic therapy.

2. Administer photodynamic therapy at primary cell site; then

Wait a period of minutes to days before administering the DETOX compound, said compound being administered at a 10% full strength concentration; and

Repeat administrations of the photodynamic therapy and/or DETOX and/or other immunologic adjuvant at intervals as required to sustain a cell specific immunologic response.

3. Administer photodynamic therapy at primary cell site; then

Wait a period of minutes to days before administering DETOX to the body; and

Administer to the body either singularly, or at intervals, an immune modulator in order to continue to promote a tumor cell specific systemic immunologic response.

While the preferred embodiments of the above method have been described in detail, it is understood that various changes and adaptations may be made without departing from the spirit and scope of the appended claims.

What is claimed is:

1. A method of treating a living body having a primary tumor and a metastatic tumor, said method comprising:
identifying a primary tumor tissue site of the living body;
combining an administration of an immunologic adjuvant at a predetermined concentration to said body, said predetermined concentration being approximately a standard concentration for immunization procedures of said body, said administered immunologic adjuvant resulting in a systemic condition of heightened nonspecific enhanced immune system of the body, including an increased level of nonspecific immune-related molecular and cellular factors and cells, and an administration of a photodynamic light therapy proximate said primary tumor tissue site, said photodynamic light therapy having a light wavelength and a sufficient light dosage to eradicate tumor cells within the primary tumor tissue site, said eradicated primary tumor tissue site cells releasing necrosis-related tumor cell specific antigens; and
promoting and enhancing a systemic immunologic response of said body as a result of an interaction between the increased level of nonspecific immune-related molecular and cellular factors and cells and photodynamic light therapy released tumor cell specific antigens, said systemic immunologic response yielding increased levels of tumor cell specific antibodies and other immunologic anti-tumor cell specific products and cells for eradicating cells of the metastatic tumor.

2. The method of claim 1 wherein the light wavelength ranges from about 400 nm to about 800 nm, the light dosage ranges from about 10 J/cm² to about 250 J/cm² and a light dosage rate ranges from about 50 mw/cm² to about 200 mw/cm².

3. The method of claim 1 wherein the wavelength ranges from about 300 nm to about 700 nm.

4. The method of claim 1 wherein the administration of the immunologic adjuvant is achieved via one or more of the group containing an intravenous injection, an injection proximate the primary tumor site, a topical administration, a subcutaneous injection, and an injection within the primary tumor site.

5. The method of claim 1 wherein the step of administering the photodynamic light therapy includes the step of administering a photosensitizing agent.

6. The method of claim 5 wherein the administration of the photosensitizing agent is achieved via one or more of the group containing an intravenous injection, an injection proximate the primary tumor site, a topical administration, a subcutaneous injection, and an injection within the primary tumor site.

7. The method of claim 1 wherein the immunologic adjuvant is DETOX.

8. The method of claim 7 wherein the DETOX is administered at a 10% full strength concentration.

9. The method of claim 1 wherein the administration of the immunologic adjuvant precedes the administration of the photodynamic light therapy.

10. The method of claim 1 wherein the administration of the photodynamic light therapy precedes the administration of the immunologic adjuvant.

11. The method of claim 1 wherein the administration of the photodynamic light therapy and the administration of the immunologic adjuvant occur at least in part simultaneously.

12. The method of claim 1 wherein the administration of the immunologic adjuvant includes one or more separate administrations of the adjuvant to the body.

13. The method of claim 1 wherein the administration of the immunologic adjuvant includes administrations before and after the administration of the photodynamic light therapy.

14. The method of claim 1 further comprising the step of:
administering of an immune modulator to the body before, during, or after the administration of photodynamic light therapy.

15. The method of claim 14 wherein the immune modulator is administered at multiple times to the body.

16. A method of eradicating acellular organisms of a living body by promoting a systemic immunologic activity, said method comprising the steps of:
identifying an acellular organism site of the living body;
combining an administration of an immunologic adjuvant to said body, said immunologic adjuvant promoting a systemic increased level of molecular and cellular factors and cells necessary to effect a systemic immunologic response and an administration of a photodynamic light therapy proximate said acellular organism site, said photodynamic light therapy having a sufficient light dosage to cause cell necrosis of an acellular organism within the acellular organism site, said photodynamic acellular organism necrosis resulting in the release of necrosis-generated acellular organism antigens; and
promoting and enhancing a systemic acellular organism-related immunologic response of said body as a result of an interaction between the molecular and cellular factors and cells and the necrosis-generated acellular organism specific antigens, said systemic immunologic response yielding an increased level of acellular organism specific antibodies and other immunologic acellular organism specific products and cells specifically targeted to immunologically eradicate similar acellular organisms.

17. The method of claim 16 wherein the administration of the immunologic adjuvant precedes the administration of the photodynamic light therapy.

18. The method of claim 16 wherein the administration of the photodynamic light therapy precedes the administration of the immunologic adjuvant.

19. The method of claim 16 wherein the administration of the photodynamic light therapy and the administration of the immunologic adjuvant occur at least in part simultaneously.

20. The method of claim 16 wherein the light wavelength ranges from about 400 nm to about 800 nm, the light dosage ranges from about 10 J/cm² to about 250 J/cm² and a light dosage rate ranges from about 50 mw/cm² to about 200 mw/cm².

21. The method of claim 16 wherein the wavelength ranges from about 300 nm to about 700 nm.

22. The method of claim 16 wherein the administration of the immunologic adjuvant is achieved via one or more of the group containing an intravenous injection, an injection proximate the acellular organism site, a topical administration, a subcutaneous injection, and an injection within the acellular organism site.

23. The method of claim 16 wherein the step of administering the photodynamic light therapy includes the step of administering a photosensitizing agent.

24. The method of claim 16 wherein the administration of the photosensitizing agent is achieved via one or more of the group containing an intravenous injection, an injection proximate the acellular organism site, a topical administration, a subcutaneous injection, and an injection within the acellular organism site.

25. The method of claim 16 wherein the immunologic adjuvant is DETOX.

26. The method of claim 16 further comprising the step of:
administering the immunologic adjuvant to the body for a period of time following the photodynamic light therapy.

27. The method of claim 16 further comprising the step of:
repeating the administrations of the photodynamic light therapy.

28. The method of claim 16 further comprising the step of:
repeating the administrations of the immunologic adjuvant.

29. The method of claim 16 wherein the acellular organism is a fungus.

30. The method of claim 16 wherein the acellular organism is a virus.

31. The method of claim 16 further comprising the step of:
administering of an immune modulator to the body before, during, or after the administration of photodynamic light therapy.

32. The method of claim 31 wherein the immune modulator is administered to the body at more than one administration.

33. A method of eradicating cellular organisms of a living body by promoting a systemic immunologic activity, said method comprising the steps of:
identifying a cellular organism site of the living body;
combining an administration of an immunologic adjuvant to said body, said immunologic adjuvant promoting a systemic increased level of molecular and cellular factors and cells necessary to effect a systemic immunologic response and an administration of a photodynamic light therapy proximate said cellular organism site, said photodynamic light therapy having a sufficient light dosage to cause cell necrosis of a cellular organism within the cellular organism site, said photodynamic cellular organism necrosis resulting in the release of necrosis-generated cellular organism antigens; and
promoting and enhancing a systemic cellular organism-related immunologic response of said body as a result of an interaction between the molecular and cellular factors and cells and the necrosis-generated cellular organism specific antigens, said systemic immunologic response yielding an increased level of cellular organism specific antibodies and other immunologic cellular organism specific products and cells specifically targeted to immunologically eradicate similar cellular organisms.

34. The method of claim 33, wherein the administration of the immunologic adjuvant precedes the administration of the photodynamic light therapy.

35. The method of claim 33, wherein the administration of the photodynamic light therapy precedes the administration of the immunologic adjuvant.

36. The method of claim 33, wherein the administration of the photodynamic light therapy and the administration of the immunologic adjuvant occur at least in part simultaneously.

37. The method of claim 33 wherein the administration of the immunologic adjuvant is achieved via one or more of the group containing an intravenous injection, an injection proximate the cellular organism site, a topical administration, a subcutaneous injection, and an injection within the cellular organism site.

38. The method of claim 33 wherein the administration of the photosensitizing agent is achieved via one or more of the group containing an intravenous injection, an injection proximate the cellular organism site, a topical administration, a subcutaneous injection, and an injection within the cellular organism site.

39. The method of claim 33 further comprising the step of:
administering the immunologic adjuvant to the body for a period of time following the photodynamic light therapy.

40. The method of claim 33 further comprising the step of:
repeating the administrations of the photodynamic light therapy.

41. The method of claim 33 further comprising the step of:
repeating the administrations of the immunologic adjuvant.

42. The method of claim 33 wherein the cellular organism is a bacteria or a parasite.

43. The method of claim 33 further comprising the step of:
administering of an immune modulator to the body before, during, or after the administration of photodynamic light therapy.

44. The method of claim 43 wherein the immune modulator is administered to the body at more than one administration.
